(19) United States
(12) Patent Application Publication
(10) Pub. No.: US 2025/0295905 A1
(43) Pub. Date: Sep. 25, 2025

(54) Endoscopic Submucosal Drug Delivery System

(71) Applicant: Carnegie Mellon University, Pittsburgh, PA (US)

(72) Inventors: Spencer Matonis, Pittsburgh, PA (US); Juan P. Reyes Genere, Saint Louis, MO (US); Vladimir Lamm, Pittsburgh, PA (US); Christopher Chang, Pittsburgh, PA (US)

(21) Appl. No.: 19/085,327

(22) Filed: Mar. 20, 2025

Related U.S. Application Data
(60) Provisional application No. 63/567,754, filed on Mar. 20, 2024.

(51) Int. Cl. A61M 39/02

(52) U.S. Cl. CPC A61M 39/0247; A61M 2039/0205; A61M 2039/0255; A61M 2039/0276; A61M 2039/0291

(57) ABSTRACT

Provided herein is a delivery system and method for delivering a depot within a submucosal pocket comprising a housing including a central opening and a fluidic connector configured to secure to a fluidic supply, an extension configured to at least partially collapse within the central opening of the housing, a supply conduit configured to extend through the housing and the extension, the supply conduit configured to deliver a lift solution configured to form a submucosal pocket within a subject, and a depot conduit configured to extend through the housing and the extension and to deposit a depot within the submucosal pocket, where when the extension is configured to at least partially collapse within the housing, whereby at least one of the supply conduit or depot conduit, or both advance distally past a distal end of an endoscope.

### CROSS-REFERENCE

This application claims the benefit of U.S. Provisional Application No. 63/567,754, filed Mar. 20, 2024, which application is incorporated herein by reference.

### STATEMENT AS TO FEDERALLY SPONSORED RESEARCH

This disclosure was made with United States government support under 2230953 awarded by the National Science Foundation. The U.S. government has certain rights in the invention.

### BACKGROUND

Intravenous medications, oral pills and subcutaneous injections (biologics) are delivered systemically in the body's bloodstream. They are distributed rapidly, but are effectively “diluted” in the patient's blood. Systemic medications are useful for systemic symptoms, but they have a hard time addressing stubborn, localized symptoms. For example, in the context of inflammatory bowel disorders (IBD), this includes cases of stricturing Crohn's Disease, perianal Crohn's and localized ulcerative colitis. These disease cases often require surgery due to the difficulties of pharmacological management. Increased doses of systemic medications are a common source of side effects as drugs end up concentrating in other parts of the body. There is a need for a treatment option that can maximize drug bioavailability, minimizes side effects, and target localized symptoms.

### SUMMARY

Another aspect of the present disclosure provides a delivery system for delivering a depot within a layer of an intestine or gastrointestinal tract, such as an intraintestinal or submucosal pocket, the delivery system may comprise a housing including a central opening and a fluidic connector configured to secure a fluidic supply; an extension configured to at least partially collapse within the central opening of the housing; a supply conduit configured to extend through the housing and the extension, the supply conduit configured to deliver a lift solution configured to form an intraintestinal or submucosal pocket within a subject; and a depot conduit configured to extend through the housing and the extension and to deposit a depot within the intraintestinal or submucosal pocket; where when the extension may be configured to at least partially collapse within the housing, whereby at least one of the supply conduit or depot conduit, or both advance distally past a distal end of an endoscope.

In an aspect, the delivery system may comprise a sheath connected to a distal end of the extension, wherein when the extension at least partially collapses within the housing, at least one of the supply conduit or depot conduit, or both may advance past a distal end of the sheath. The sheath may extend along at least a portion of the endoscope. The delivery system may comprise a distal connector configured to be secured to the extension and the sheath. The distal connector may be configured to provide rigidity to prevent kinking of the sheath. The sheath may be friction fit to the distal connector. The sheath may be screwed to the distal connector. The distal connector may be partially housed within the extension. The distal connector may be secured to a distal end of the extension.

In an aspect, the delivery system may comprise a trigger configured to secure the extension relative to the housing. The extension may include a set of extrusions configured to interface with the trigger and prevent and/or promote advancement of the extension relative to the housing. The trigger may include a spring to maintain engagement of the trigger to the extension. In non-limiting embodiments, a knob may be used for the trigger. In non-limiting embodiments, the trigger may be configured for controlled and reversible advancement of the extension relative to the housing. In non-limiting embodiments, the delivery system may be configured to lock the needle position and lock the maximum extension range of the needle.

In an aspect, the housing may include a supply conduit recess and or a depot conduit recess. The supply conduit recess may be configured to at least partially direct the supply conduit towards an extension lumen of the extension and/or a distal connector. The depot conduit recess may be configured to at least partially direct the depot conduit towards an extension lumen of the extension and/or a distal connector.

In an aspect, the delivery system may comprise an endoscope connector configured to secure the delivery system to the endoscope. The endoscope connector may be secured to the distal end of the extension. The endoscope connector may be secured to a distal connector. The supply conduit may be concentric with the depot conduit.

In an aspect, a distal tip of the supply conduit may be staggered from the distal tip of the depot conduit.

In an aspect, the depot may be a filament or a pellet. The depot may be a series of depots configured to advance inline. The depot may be a continuous filament configured to curl within the intraintestinal or submucosal pocket. The depot may include one or more extra-luminal pull-tabs. The depot may include two or more depots have differing Young's Modului. The depot may include two or more depots have differing doses of a same drug or a different drug. The depot may include a programmed depot release cadence of greater than 3 months. The depot may include a programmed degradation window of greater than 3 months. The depot may include a programmed depot release cadence having more than one crest in delivered drug concentration. The housing may include a second fluidic supply configured to hold at least one of a drug and an activation agent.

In an aspect, the delivery system may comprise a loading chamber configured to receive the depot or a depot cartridge. The loading chamber may include a cartridge opener configured to receive or to open the depot cartridge in a sterile manner. The housing may be configured to secure to a fluidic supply and be in communication with the supply conduit. The fluidic supply may be removable. The fluidic supply may be integrated within the housing. The fluidic supply may be a syringe. The fluidic supply may be released using a controller. The fluidic supply may be manually released.

Another aspect of the present disclosure provides a method for delivering a depot into a portion of submucosa of a subject comprising loading a depot in a delivery system comprising a supply conduit, a depot conduit, a housing, and an extension, the depot conduit and the supply conduit being configured to extend to a distal tip of an endoscope; advancing the depot conduit and the supply conduit approximate an insertion site at the portion of submucosa of the subject; advancing a distal end of the supply conduit into a portion of submucosa of the subject; forming an intraintestinal or submucosal pocket within the subject; and delivering the depot through the depot conduit and into the intraintestinal or submucosal pocket.

In an aspect, the depot conduit and the supply conduit are advanced through a sheath connected to a distal end of the extension. In an aspect, delivering the depot may be done without removing the distal end of the supply conduit from the submucosa of the subject. In an aspect, forming the intraintestinal or submucosal pocket within the subject may include delivering a lift solution through the supply conduit into the submucosa of the subject. In an aspect, the supply conduit may be configured to deliver the lift solution within a fluidic supply secured to the housing. In an aspect, the lift solution may be configured to be retained for about 10 minutes. In an aspect, the lift solution may be configured to be retained for about one (1) hour. In an aspect, the depot may be delivered using a second fluidic supply secured to the housing.

Another aspect of the present disclosure provides a method for delivering a depot into a submucosa of a subject, the method comprises: loading a depot in a delivery system comprising a housing, an extension, a supply conduit in communication with a fluidic supply, and a depot conduit, the depot conduit and the supply conduit configured to extend to a distal tip of an endoscope; advancing the depot conduit and the supply conduit approximate an insertion site at submucosa of the subject by moving the housing relative to the extension; forming an intraintestinal or submucosal pocket within a portion of submucosa of the subject using the fluidic supply; and delivering the depot through the depot conduit and into the submucosal pocket.

Another aspect of the present disclosure provides a system for delivering a subcutaneous depot, the system comprises: an endoscope comprising a working channel; a fluidic supply; a delivery system for delivering a depot within the intraintestinal or submucosal pocket comprising a housing including a central opening and a fluidic connector configured to secure to the fluidic supply, an extension configured to at least partially collapse within the central opening of the housing, a supply conduit configured to extend through the housing and the extension, the supply conduit configured to deliver a lift solution configured to form an intraintestinal or submucosal pocket within a subject, and a depot conduit configured to extend through the housing and the extension and to deposit a depot within the intraintestinal or submucosal pocket, wherein when the extension at least partially collapses within the housing, at least one of the supply conduit or depot conduit, or both advance distally past a distal end of the endoscope.

Another aspect of the present disclosure provides a method for delivering a depot into a submucosa of a subject, comprising loading a depot in a depot conduit of a delivery system comprising a supply conduit, a housing, and an extension, wherein when the extension may be positioned in an extended position from the housing, the supply conduit and the depot conduit are retracted relative to a distal end of an endoscope; advancing the housing towards the distal end of the extension, where the supply conduit and/or the depot conduit are extended relative to the distal end of the endoscope; advancing the supply conduit into a submucosa of a subject; introducing a lift solution from a fluidic supply in communication with the supply conduit into the submucosa of a subject, where the lift solution may be configured to form an intraintestinal or submucosal pocket within the subject; and delivering the depot through the depot conduit and into the submucosal pocket. The method may include delivering the depot through the depot conduit and into the intraintestinal or submucosal pocket within the subject may be done without removing the distal end of the supply conduit from the submucosa of the subject.

Another aspect of the present disclosure provides a method for delivering a depot into a submucosa of a subject, the method may comprise: loading a delivery system having a housing, an extension, a depot conduit, and a fluidic conduit having a conduit tip into a working channel of an endoscope and clip the delivery system into place at a biopsy port; locking the extension and the housing in a retracted position; securing a fluidic supply holding submucosal lift solution into an adapter in communication with the housing and the fluidic conduit; identifying an injection site proximal to symptomatic tissue; advancing the conduit tip at a shallow angle into the submucosal tissue by moving the housing relative to the extension; injecting the lift solution into the submucosa to create a temporary fluidized weal; loading with the conduit tip still in the temporary fluidized weal, a depot into the depot conduit, advancing a stylet behind the depot to advance the depot to a distal end of the depot conduit; implanting the depot into the temporary fluidized weal; withdrawing the stylet and retract the housing; and removing the delivery system from the endoscope and the endoscope scope from the subject.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A illustrates a submucosal delivery system depositing a depot through an endoscope in a submucosa layer of an intestine according to some implementations.

FIG. 1B illustrates a submucosal delivery system including a housing, an extension, a supply conduit, and a depot conduit, the supply conduit and the depot conduit being configured to extend through an endoscope, to form a submucosal cushion within a submucosa layer, and to deliver a depot within the submucosal cushion according to some implementations.

FIG. 1C illustrates an endoscope having a working channel according to some implementations.

FIG. 1D illustrates a cross-section of an endoscope working channel encompassing a sheath encompassing a supply conduit and a depot conduit according to some implementations.

FIG. 1E illustrates a cross-section of an endoscope working channel encompassing a sheath encompassing a supply conduit concentric with a depot conduit according to some implementations.

FIGS. 2A-2B illustrate anatomical references of intestinal layers.

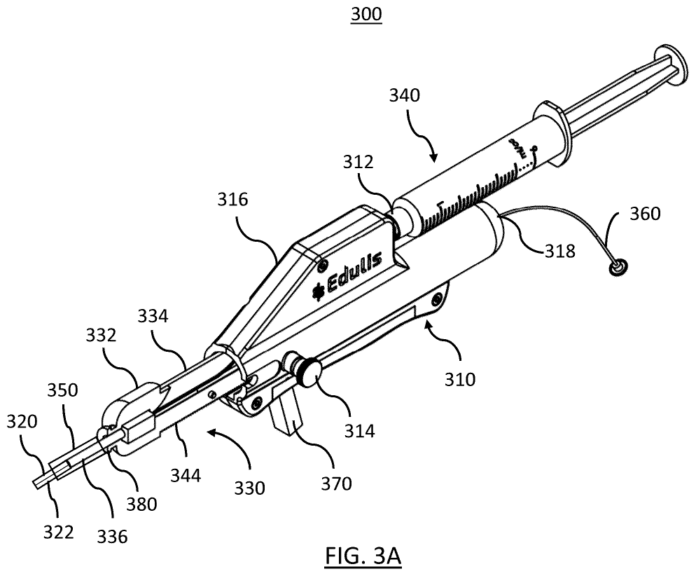

FIG. 3A illustrates a delivery system assembled with a fluidic supply including a lift solution according to some implementations.

FIG. 3B illustrates the delivery system of FIG. 3A disassembled according to some implementations.

FIG. 3C illustrates a cross section of a delivery system according to some implementations.

FIG. 3D illustrates a delivery system including the extension collapsed within the housing, where the supply conduit and the depot conduit are extended from the distal end of the extension according to some implementations.

FIG. 3E illustrates the delivery system of FIG. 3D with the extension extending from the housing, whereby the supply conduit and the depot conduit are sheathed within the distal end of the extension according to some implementations.

FIG. 4A illustrates a cross section of a delivery system including a trigger configured to releasably secure the extension to the housing according to some implementations.

FIG. 4B illustrates a cross section of a delivery system including a stylet guard according to some implementations.

FIG. 4C illustrates a delivery system including a rolling mechanism configured to control movement of the extension relative to the housing according to some implementations.

FIG. 4D illustrates a delivery system including a payload drive system for self-resetting and/or advancement of a filamentous depot or payload and/or the stylet according to some implementations.

FIG. 4E illustrates a delivery system including a loading chamber or loading latch configured to receive a depot or a depot cartridge according to some implementations.

FIGS. 4F-4G illustrate a depot cartridge according to some implementations.

FIGS. 5A-5I illustrate various views of a delivery system according to some implementations.

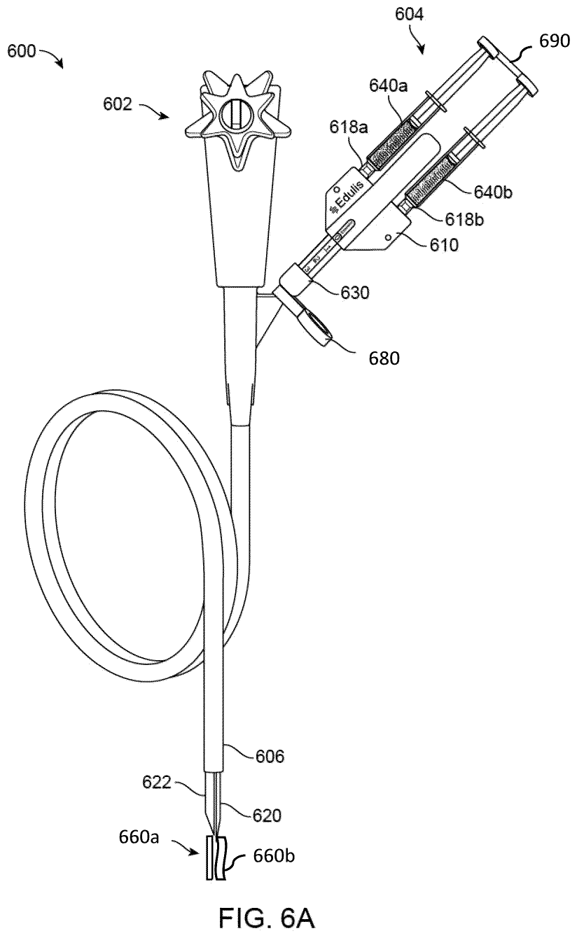

FIGS. 6A-6C illustrate a delivery system configured to secure at least two fluidic supplies configured for depositing a depot through an endoscope in a submucosa layer of an intestine according to some implementations.

FIGS. 7A-7C illustrate methods of using a delivery system to deliver a depot according to some implementations.

FIGS. 8A-8F illustrate motion of a delivery system delivering a depot according to some implementations.

FIG. 9A illustrates a delivery system including a supply conduit and a depot conduit configured to protrude staggered from a sheath extending to a distal end of an endoscope according to some implementations.

FIG. 9B illustrates a delivery system including a supply conduit and a depot conduit configured to protrude simultaneously from a sheath extending to a distal end of an endoscope according to some implementations.

FIG. 10 illustrates a series of stages in a method of delivering a depot and treating an underlying condition according to some implementations.

FIG. 11A illustrates a series of depots configured to advance inline according to some implementations.

FIG. 11B illustrates a continuous filament according to some implementations.

FIG. 11C illustrates a continuous filament including at least one segment having differing Young's Modulus according to some implementations.

FIG. 11D illustrates a depot having one or more extra-luminal pull-tabs or retrieval loops.

FIG. 12 illustrates a drug release cadence from the delivery system compared to traditional therapeutic windows according to some implementations.

FIG. 13 shows a graph of an average drug concentration benchtop degradation study over a month for two types of depots.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

In an aspect, described herein are delivery systems and methods for depositing a depot or medical payload in a layer of an intestine or gastrointestinal tract. In some implementations, the depositing is done within a submucosa layer. In some implementations, the system described herein may be configured for sustained and/or localized administration of a depot including small molecule therapeutics. In some embodiments, the system may be used for treating diseases such as inflammatory bowel disease (IBD), which includes Crohn's disease (CD) and ulcerative colitis (UC). In some embodiments, the depots may include analgesics. In an example, analgesics may include for management of abdominal and/or post-operative pain. In some embodiments, the system may be used in delivering therapeutics for appetite suppression or weight loss. In some embodiments, the system may be used for delivering cancer treatments including radiotherapeutics for brachytherapy. In some embodiments, the depot may include medical payloads comprising drugs and/or devices.

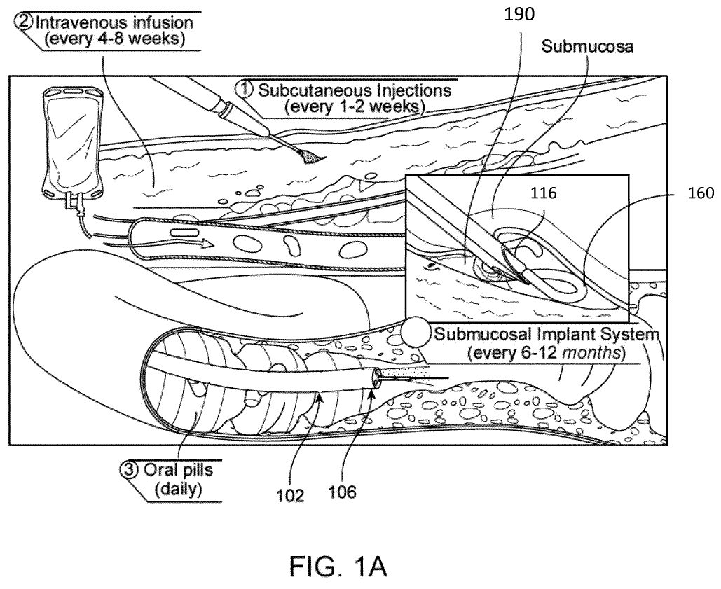

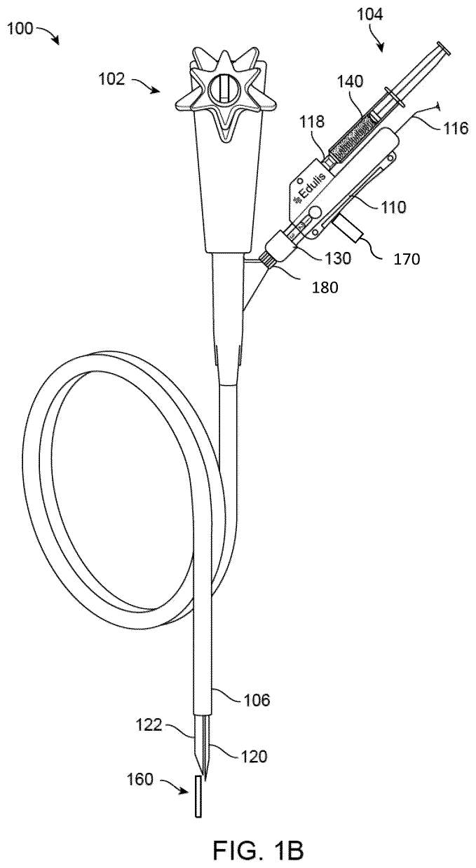

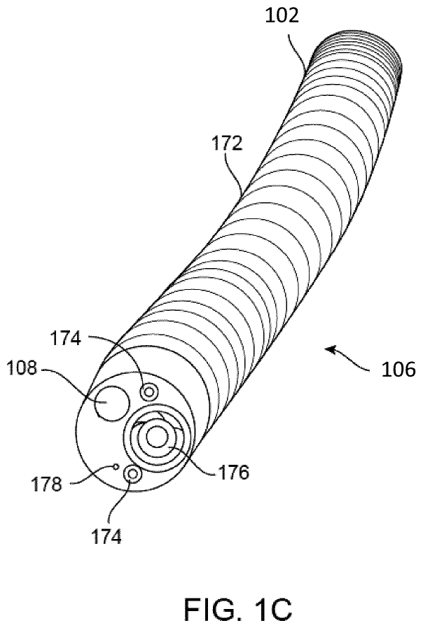

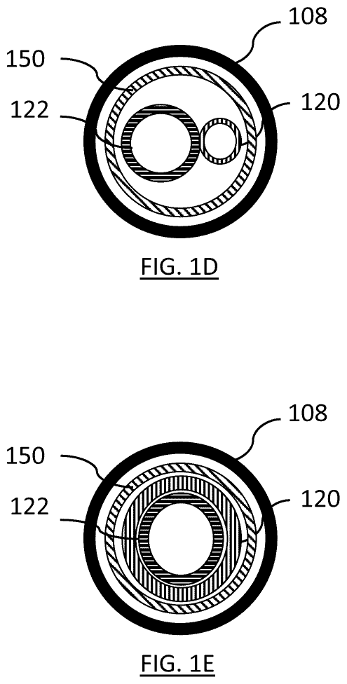

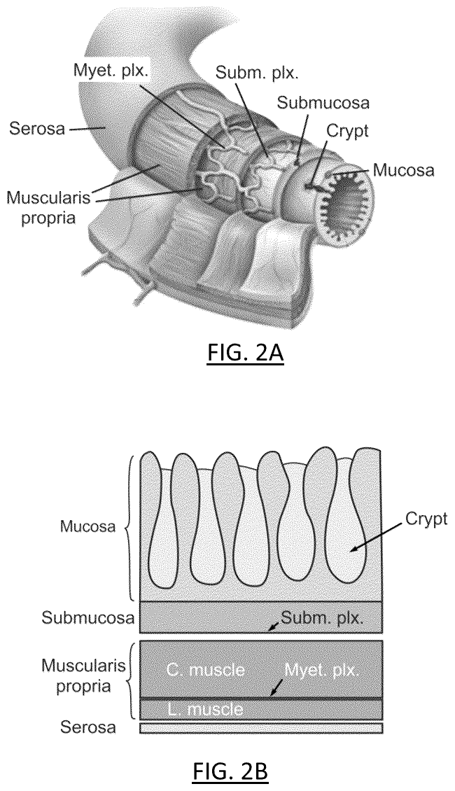

FIG. 1A illustrates an embodiment of a submucosal implant system as compared to existing modes of drug administration, subcutaneous injections, intravenous infusion, oral pills, along with examples of their respective therapeutic windows, daily pills, and monthly infusions. In some embodiments, the depot may be inserted into an intraintestinal or submucosal pocket and/or fluidized cushion. In an aspect, the intraintestinal or submucosal pocket may be a cushion or lift created to accommodate the depot. In some embodiments, the intraintestinal or submucosal pocket may provide a fluidized cavity, in which an elongated depot may be curled into. In some embodiments, the intraintestinal or submucosal pocket may be formed using submucosal lift techniques. In some implementations, the intraintestinal or submucosal pocket and/or fluidized cushion formed using a lift solution 190. Examples of lift solutions may include saline or a non-saline lift solution, a non-water-based lift solution, an oil-based lift solution, a hydrophobic solution configured to slow down permeation of water.

In some embodiments, the delivery system may be adapted to fit into an endoscope, colonoscope, and/or enteroscope. Examples of endoscopes may include Exera II & III (Olympus America Inc). Turning to FIG. 1B, a system 100 is illustrated including an endoscope 102 and a delivery system 104 configured to deliver a depot 160 in a submucosa layer of an intestine or gastrointestinal tract according to some implementations. In some implementations, the delivery system 104 may include a housing 110 and an extension 130 configured to at least partially collapse within the housing. In some implementations, the delivery system may include a supply conduit 120 configured to form a submucosal cushion within or adjacent to the submucosa layer and facilitate administration of the depot. In an example, the supply conduit 120 may include a sharpened tip or needle on a distal end to facilitate penetration. In an example, the supply conduit 120 may be an elongated saline needle. In some implementations, the delivery system may include a depot conduit 122 configured to extend through the housing and the extension. In an aspect, the depot conduit may be used to advance the depot to the submucosal cushion. In an example, the depot conduit may be an elongated filament needle. In an example, the supply conduit and the depot conduit may have a working distance length of about 90-250 cm. In an example, the supply conduit may be a 22-gauge needle. In an example, the depot conduit may be a 20-gauge needle. In an example, the supply conduit and the depot conduit may be 304 SS ultra-thin wall hypodermic tubing.

In some embodiments, the delivery system 104 may include an endoscope connector 180 configured to mate to standard endoscope and/or colonoscope instrument channel ports. In some implementations, the delivery system may include an endoscope having a working channel 108 (FIG. 1C). In some implementations, the system may include an endoscope having one or more of an insertion tube having a bending section 172, an illumination lens 174, an objective lens 176, and a gas, fluid, and a suction nozzle 178. In some implementations, the system may include a heater configured to warm the depot. In an example, the heater may be part of the endoscope. In an example, the heater may be part of the delivery system.

In some implementations, the system may include a sheath 150 configured to enclose at least one of the supply conduit 120 or the depot conduit 122, or both (FIG. 1C). In an example, the sheath 150 may connect at a distal end of the extension 130 and extend to a distal end 106 of the endoscope. In some implementations, the sheath may be configured to have a working length tailored to a corresponding endoscope. In some implementations, the sheath may be configured to pass through standard endoscope and/or colonoscope instrument ports. In an example, a working channel may be about 2.4 mm or greater in diameter. In an aspect, the lengths of the supply conduit and depot conduit are tailored/scaled to the working channel length of the endoscope in use. Typical working channel lengths across endoscopes range from 90-250 cm, with the supply conduit and the depot conduit being approximately 20-30 cm longer in total. In an example, a length of a gastroscope may be about 90 cm. In an example, a length of an enteroscope or colonoscope may be about 230 cm. In an aspect, the supply conduit and the depot conduit maybe configured to be up to about 1 cm more proximal to the distal end of the working channel to up to about 5 cm more distal to the distal end of the working channel. In an aspect, the supply conduit and the depot conduit maybe configured to be retracting to a position about 0-1 cm beyond a fully retracted position within the endoscope, the working channel, and/or the sheath. In an aspect, the supply conduit and the depot conduit maybe configured to extend up to 1-5 cm beyond the end of the endoscope, the working channel, and/or the sheath.

In some implementations, the supply conduit may be secured (e.g., securely attached) at least in part to the depot conduit. In an example, a distal end of the supply conduit may be adhered to a distal end of the depot conduit. In an example, the supply conduit and the depot conduit may be partially secured to each other using a fastener, an adhesive, welding, or other means. In an example, the supply conduit and the depot conduit may be extruded from a common or hybrid material. In an example, the depot conduit may be a tubing. In an aspect, securing the supply conduit and the depot conduit may still allow for flexing to accommodate movement of the extension. In an aspect, the supply conduit and the depot conduit allow for flexibility in the endoscope and delivery system to allow for navigation of the bowel.

In some implementations, the distal end of the supply conduit may be offset in distance from the distal end of the depot conduit. In an example, the offset may range from about 0-10 mm. In an example, the offset may be about 100 μm. In an example, the offset may be about 1 mm.

In some implementations, the working channel and/or the sheath may include an anti-stick coating on catheter sheathing for smooth passage through the working channel. In some implementations, catheters and/or conduits may include a low friction coating on their exterior surface. In an example, low friction coating may be hydrophilic material such as PEG, PVP, or hydrogel configured to create lubricity in presence of liquid. In an example, low friction coating may include PTFE fluoropolymer or plasma-deposited parylene. In some implementations, the sheath, the depot conduit, and/or the supply conduit may include materials such as PTFE, stainless steel, PEEK, nitinol, coiled metal, rubber, etc. In some implementations, the system may include catheter layers and/or materials which at least partially restrict a bending radius of the endoscope.

In some implementations, the supply conduit may be secured to the depot conduit (FIG. 1D). In some embodiments, the delivery system may include concentric and/or adjacent conduits. In an example, the supply conduit may be concentric with the depot conduit (FIG. 1E). In some embodiments, the delivery system may include a concentric conduit, which may be configured for single-operation cushion and/or insertion. In embodiments using a concentric (or nested needle) conduit, the depot conduit may run through the supply conduit line. In an example, the concentric conduit may be a double chambered needle where a first chamber of the double chambered needle may be used for delivering the lift solution and a second chamber of the double chambered needle may be used for delivering the depot. In an aspect, having a fluidized channel or supply conduit outside of a payload channel or depot conduit may be beneficial for several reasons. In an example, the user may only have to locate the submucosa once before injecting lift solution and/or implant. In an example, benefits may include not requiring a swap of syringes, not requiring re-entry, and only requiring one perforation. In an example, the conduit tip may be introduced to the tissue at a shallow angle. In an aspect, the conduit tip may be tuned to have a stiffness hard enough to penetrate the submucosa but soft enough to prevent penetration into muscle. In an aspect, concentric conduits may allow for single digit actuation of the depot conduit, supply conduit, and the stylet. In an example, the handle may include individual controls or buttons configured to control advancement of the depot conduit, supply conduit, and the stylet. In an example, the controls may be configured to require a particular order of deployment such as the supply conduit then the depot conduit and then the stylet.

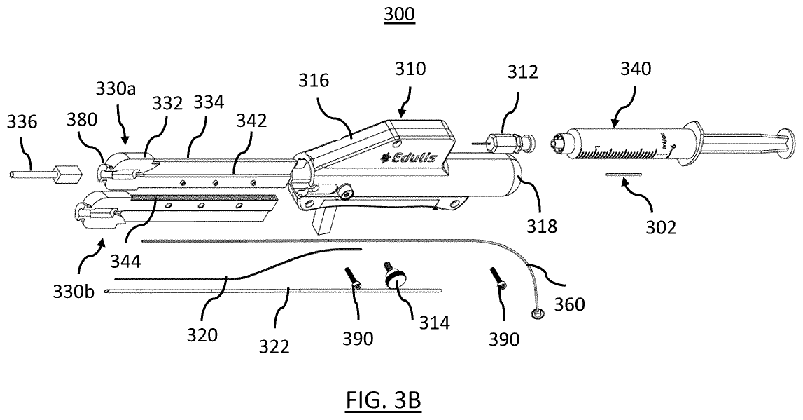

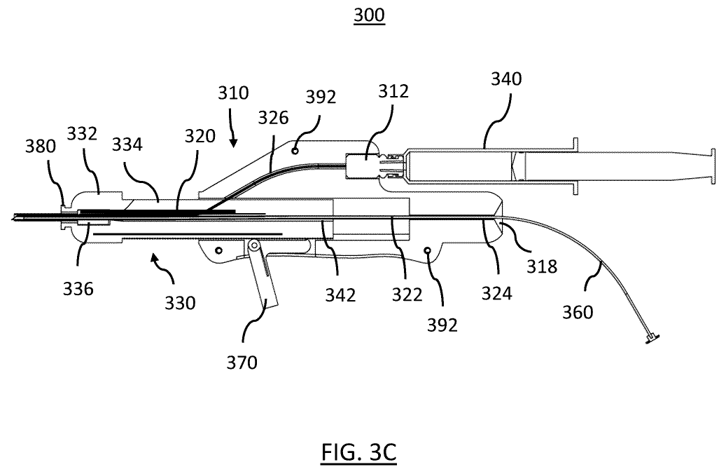

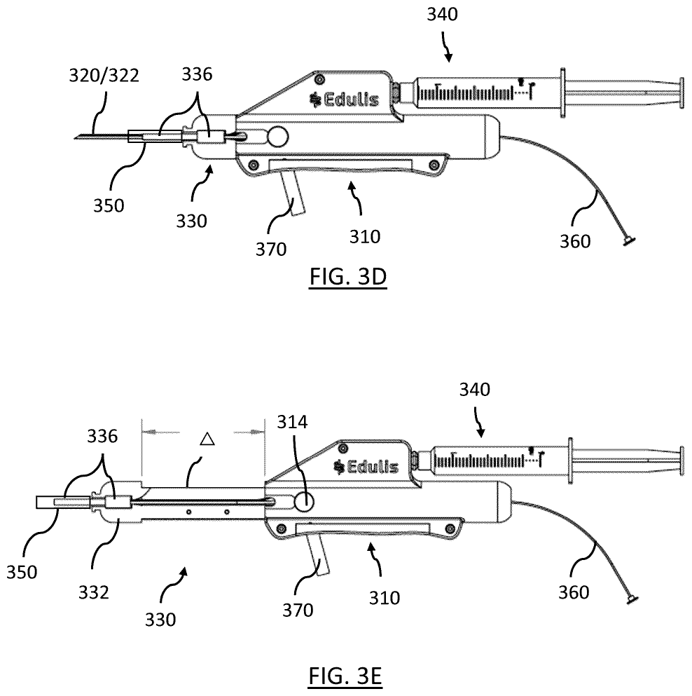

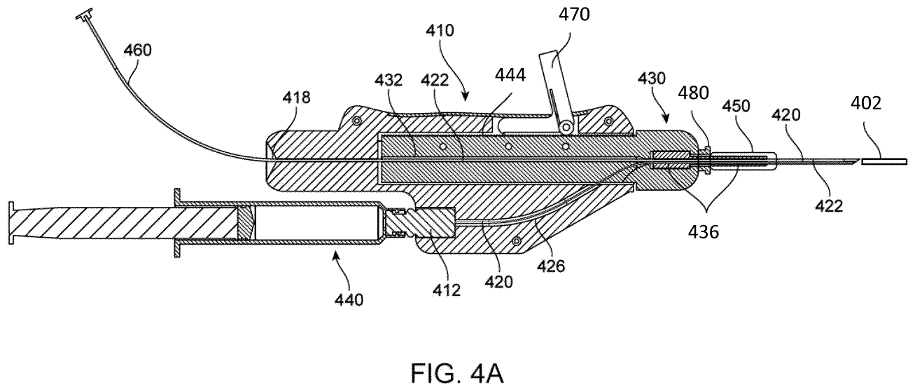

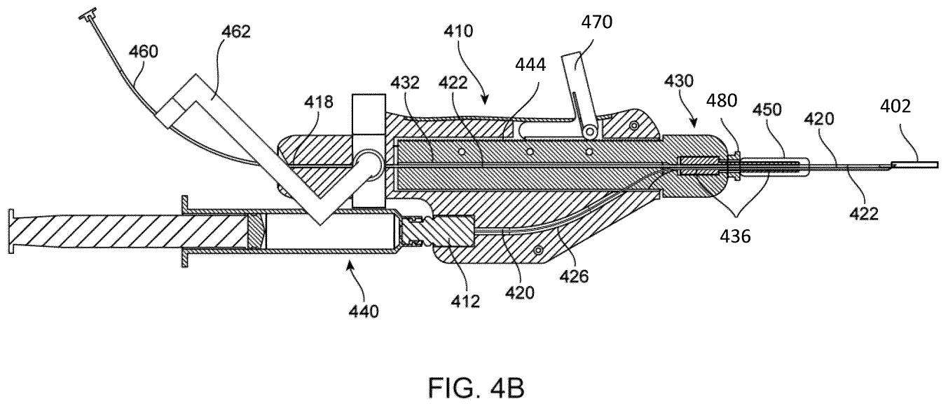

Turning to FIGS. 3A-3E, a delivery system 300 is illustrated including a housing 310 and an extension 330 configured to at least partially collapse within the housing, a supply conduit 320 configured to extend through the housing and the extension, a depot conduit 322 configured to extend through the housing and the extension and to deposit a depot. In some implementations, the delivery system or housing may include a fluidic connector 118, 312, 412 configured to secure to a fluidic supply 140, 340, 440 in communication with the supply conduit. In an example, the fluidic connector 312 may be a luer connector or supply tube fitting. In an example, the luer connector may be a slip luer connector or a twist-lock luer connector. In an example, the fluidic connector may be a pressure fit adaptor, screw on adaptor, or any other type of fluid connector. In some implementations, the delivery system or the housing may include a flange 316. In an aspect, the flange 316 may include a supply conduit recess 326 at least partially housing the fluidic connector 312 and the supply conduit 320. In some implementations, the system may include o-rings and/or gaskets for containment of injection fluid within the depot conduit and/or the supply conduit. In some implementations, the system may include catheter junctions for replaceable tubing, which may allow for a re-usable housing and/or extension. In some implementations, the fluidic supply may be a syringe or reservoir configured to hold the lift solution. In an aspect, the supply conduit 320 may be configured to extend through the housing and the extension. In an example, the fluidic supply may be removable. In an example, the fluidic supply may be integrated within the housing.

In some implementations, the depot conduit 322 may be configured to extend through the housing and the extension 330. In some implementations, the housing may include a conduit guide configured to guide at least one of the supply conduit 320 and the extension 330. In an example, the conduit guide may guide the supply conduit 320 through the housing towards the extension 330. In an example, the supply conduit 320 may be made from a tygon, silicone, or plastic material. In some implementations, when the extension 330 is collapsed within the housing, the supply conduit and/or the depot conduit are extended beyond a distal end of the extension 330 (FIG. 3D). In some implementations, when the extension 330 is extended from the housing, the supply conduit and/or the depot conduit are retracted from the distal end of the extension 330 (FIG. 3E).

In some implementations, the delivery system may include a sheath 350 configured to enclose at least one of the supply conduit 320 or the depot conduit 322, or both (FIG. 3A).

In some implementations, the housing may include a port 318, 418 to a housing lumen 324. In some implementations, the port 318 may be configured to receive a stylet 116, 360, 460. In an aspect, the port may be configured to load a depot 302, 402 in the depot conduit. In an aspect, the port may be configured to receive the stylet to advance the depot. In an example, the extension 330 may include an extension lumen 342 configured to extend from a proximal end to a distal end or endoscope connector 380. In an example, the depot conduit of the housing may be aligned with the extension lumen 342 of the extension 330. In an example, the proximal end of the extension 330 may abut within the opening of the housing. In some implementations, the stylet may be shaped to advance the depot through the depot conduit 322 and the extension lumen 342.

In an example, the depot may include a filament configured to fit within the depot conduit. In some embodiments, filaments may vary in diameter, length, additive type, and/or additive concentration. In some embodiments, drugs may be embedded within the filament in powder form. In some embodiments, drugs may be embedded within the filament at elevated temperatures.

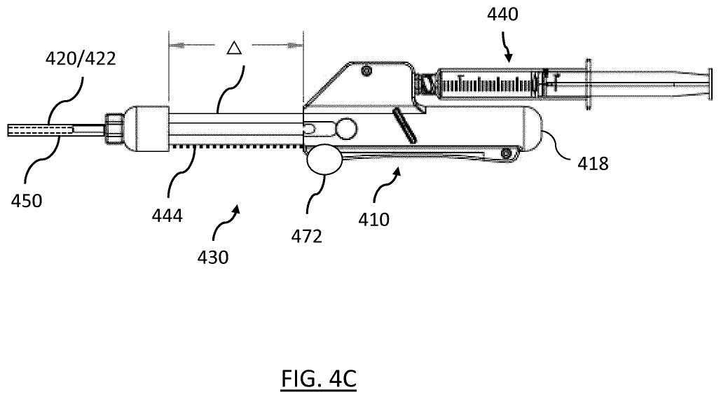

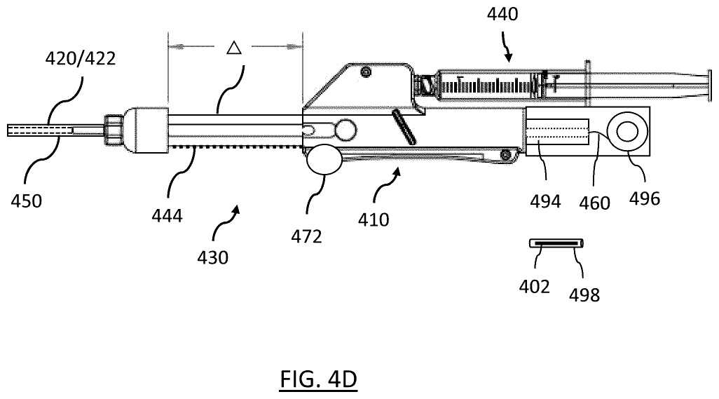

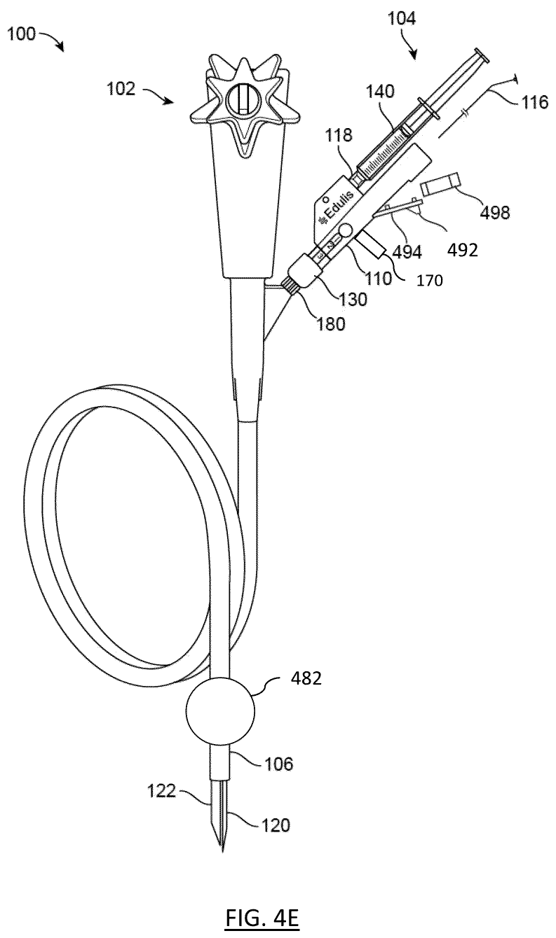

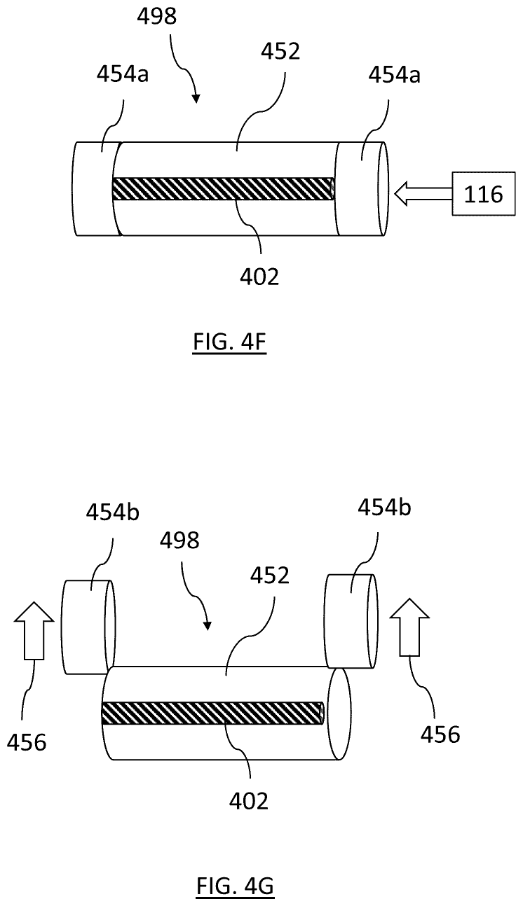

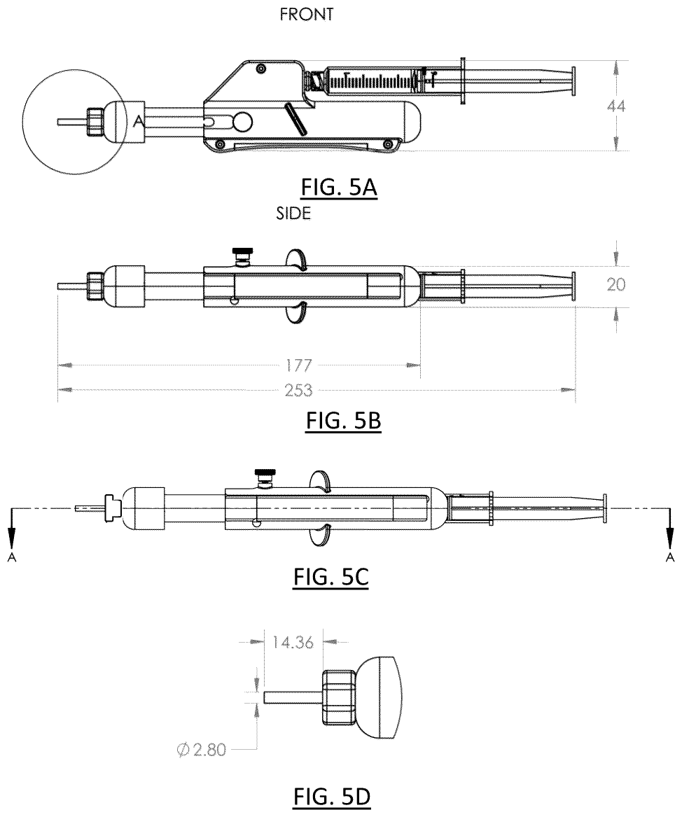

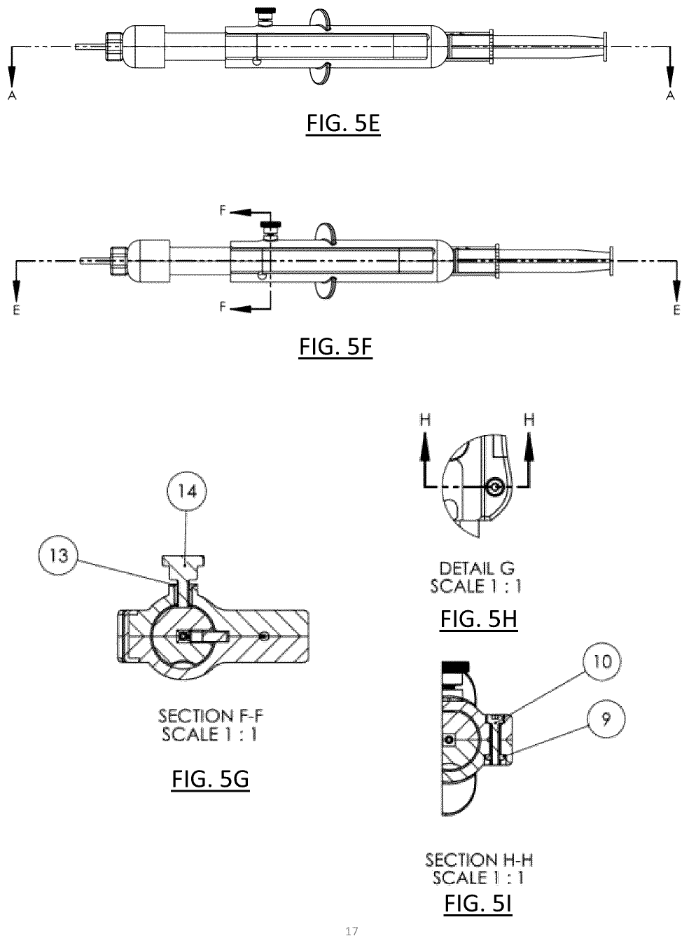

In some implementations, the delivery system may include a loading chamber or a loading latch 494 configured to receive a depot or a depot cartridge 498 (FIGS. 4D-4G). In an example, the depot cartridge may include a narrow rigid vial 452 having one or more seals 454a-b configured to be opened or punctured and exposing the depot 402. In an example, the seal may be a foil seal. In an example, the depot cartridge may be twisted to be opened. In an aspect, the depot cartridge may be configured to keep the depot sterilized. In an aspect, the depot cartridge may be configured to prevent the depot from bending in manual handling. In an example, seal may be a foil configured to be punctured. In an example, seal 454b may be a gasket configured to be pushed out of place 456. In some implementations, the seal may be opened using the stylet. In some implementations, the housing may include a protrusion configured to open the seal by puncturing or pushing the seal out of place.

The loading chamber may include a cartridge opener configured to receive or to open the depot cartridge in a sterile manner. In some implementations, the housing may include a stylet guard 462 configured to move or tilt the stylet away from the port. In an example, the stylet guard 462 may be configured to expose the depot conduit and allow for the loading of the depot, the filament, or the depot cartridge. In some implementations, the loading latch 494 may be configured for loading of solid payloads into the payload catheter via a chamber extending from the side of the housing. In an example, the loading latch may include an articulated button 492. In an example, the articulated button may open the loading latch. In an example, the loading latch may expose a portion of the housing. In an example, the loading latch may be part of the payload drive system attached or in communication with the housing. In some implementations, the system may include an articulated body for large payload loading.

In some implementations, the housing and the extension may include one or more extrusions configured to prevent advancement of the extension from advancing into or extending from the housing. In an example, the housing may include a limiter 314 configured to at least partially extend through the extension and the housing. In an example, the housing and/or the extension may include one or more extrusions configured to abut to limit motion. In some implementations, the extension 330 may include a head 332 and a body 334 connected to the head. In some implementations, the extension 330 may include an endoscope connector. In an example, the endoscope connector may include a fringe configured to mate with a biopsy port. In an aspect, the head may have a portion larger than the body, where the head prevents the extension 330 from advancing into the housing. In some embodiments, the housing may include a hard stop, a spring, and/or a dashpot feature. In some implementations, the housing may include single-direction cleats and/or spring dampeners for tactile feedback and standardization of implant insertion pressure and stylet extension, such as a dashpot. In an example, the hard stop may be configured to prevent overextension of the supply conduit and/or the depot conduit. In an example, the hard stop may be configured to prevent extension beyond the submucosa layer of tissue and/or may allow for controlled retraction. In an example, the hard stop may be configured to standardize an applied user pressure and/or prevent overextension of the supply conduit and/or the depot conduit.

In some implementations, the extension 330 may be include two or more portions configured to be assembled. In an example, the extension 330 may be include a first portion 330a and a second portion 330b configured to be secured to the first portion (FIG. 3B). In an example, the first portion and the second portion may be secured to each other using one or more fasteners 390 at one or more junctions 392. In some implementations, the extension 330 may be a unibody piece. In an example, the unibody piece may be injection molded.

In some implementations, one or more of the supply conduit 320 or the depot conduit 322, or both may be exchanged with the housing and/or the extension. In an example, supply conduits and depot conduits having different lengths and/or lumen diameters may be exchanged within the delivery system. In an example, the delivery system may include one or more push-connect and/or lock-luer fittings configured to secure the supply conduit or the depot conduit within the delivery system.

In some implementations, the extension may be secured to the housing. In some implementations, a delivery system may include a trigger 170, 370, 470, 870 configured to releasably secure the extension relative to the housing according to some implementations (FIGS. 3A, 4A). In some implementations, the trigger may include a locking mechanism or nested and/or adjacent trigger systems for single-hand user actuation of deployment triggers/plungers or stylets. In some implementations, the extension may include a plurality of extrusions 344, 444 configured to interface with the trigger and prevent and/or promote advancement of the extension relative to the housing. In some implementations, the trigger may include a spring to maintain engagement of the trigger to the extension.

In some implementations, a delivery system may include a rolling mechanism 472 configured to control movement of the extension relative to the housing (FIG. 4C). In an example, the rolling mechanism may include complementary teeth on the housing and the extension. In an example, the rolling mechanism may include a worm wheel and a worm gear. In an example, the housing may include at least one of the worm wheel and the worm gear, with the extension including a complementarity worm wheel and worm gear. In an example, the rolling mechanism may serve as a dial for fine extension of the stylet. In an example, the rolling mechanism may include a rough advancement stage and a fine advancement stage. In an example, the rolling mechanism may include fine movement for precise advancement. In some implementations, the delivery system may include a hard stop configured for manual insertion of the stylet along a first portion of the working channel and a rolling mechanism configured to advance the depot a second portion of the working channel. In an example, the first portion of the working channel may be about 90-95% of the working channel length and the second portion may be the remaining length of the working channel length. In an example, the hard stop may be a feature of the stylet itself such as a bulge, a notch, or diameter changes.

Turning to FIG. 4A, a delivery system is illustrated including a housing 410, an extension 430 configured to at least partially collapse within the housing, a supply conduit 420 configured to extend through the housing and the extension, a depot conduit 422 configured to extend through the housing and the extension and to deposit a depot 402. In some implementations, a delivery system may be used in conjunction with an endoscope comprising a working channel. In some implementations, when the extension 430 at least partially collapses within the housing, at least one of the supply conduit or depot conduit, or both, may advance past a distal end of the endoscope. In some implementations, a delivery system may include a sheath 450 connected to a distal end of the extension. In some implementations, the sheath 450 may be configured to enclose at least one of the supply conduit 420 or the depot conduit 422, or both (FIG. 4B). In an example, the sheath may extend an entire length of the working channel of the endoscope. In an example, the sheath may extend at least a portion of the working channel of the endoscope. In some implementations, when the extension 430 at least partially collapses within the housing, at least one of the supply conduit or depot conduit, or both may advance past a distal end of the sheath.

In some implementations, a delivery system or extension 330 may include a distal connector 336, 436. In an example, the distal connector may be configured as an endoscope connector. In some implementations, the distal connector may be configured to be secured to the extension and the sheath. In an example, the distal connector may be configured to provide rigidity to prevent kinking of the sheath. In an example, the sheath may be friction fit or screwed to the distal connector. In some implementations, the distal connector may be partially housed within the extension. In some implementations, the distal connector may be secured to a distal end of the extension.

In some implementations, the housing may include a supply conduit recess 426. In an aspect, the supply conduit recess may be configured to at least partially direct the supply conduit towards the extension lumen of the extension and/or the distal connector. In some implementations, the supply conduit recess may be configured to direct the supply conduit within the extension. In an example, the supply conduit recess may be configured to direct the supply conduit towards the endoscope connector or the extension lumen and/or the distal connector. In some implementations, the housing may include a depot conduit recess 432. In an aspect, the depot conduit recess may be configured to at least partially direct the depot conduit towards the extension lumen and/or the distal connector.

In some implementations, a delivery system may include an endoscope connector 480 configured to secure the delivery system to the endoscope. In an example, the endoscope connector may be secured to the distal end of the extension. In an example, the endoscope connector may be secured to the distal connector. In some implementations, the endoscope connector may be a flange configured to be secured to a complementary connector of an endoscope. In an example, the endoscope connector may be threaded. In an example, the endoscope connector may prevent rotation of the delivery system relative to the endoscope.

In some implementations, the delivery system may be used in conjunction with an endoscopic procedure using one or more balloons 482 for positioning and/or anchoring or Balloon Enteroscopy. In an example, Single-Balloon Enteroscopy (SBE) may use one inflatable balloon to advance the endoscope into the small intestine. In an example, Double-Balloon Enteroscopy (DBE) may use two alternating balloons for deep small bowel access. Examples of endoscopes may include Olympus SIF-Q180 (SBE) and Fujifilm EN-580T (DBE). In some implementations, Balloon Enteroscopy my allow for stabilizing the endoscope in the bowel such that the delivery system does not get pushed out or jostled.

In some implementations, the delivery system may include a payload drive system 496 for self-resetting and/or advancement of a filamentous depot or payload and/or the stylet 460 (FIG. 4D). In an aspect, the payload drive system 496 may be configured for auto-advancement and/or auto recoiling of a stylet spool. In some implementations, the payload drive system may include a mechanical grip and advancement feature. In some implementations, the payload drive system may include two or more opposing cams for gripping and advancing the depot. In some implementations, the payload drive system may include a mechanical mechanism configured to provide incremental advancement of the depot. In an example, the payload drive system may include a side-knock mechanism. In an aspect, the side-knock mechanism may include a barrel having an opening in a side wall. A knock lever may be mounted within the opening and pivotally mounted with respect to the barrel. The knock lever may be connected with a chuck mechanism such that by depressing the lever, the chuck mechanism may be opened to allow advancement of a piece of depot. In an aspect, depressing the lever may advance the depot in step-like equal increments. In an example, the payload drive system may include a push-button mechanism. In an example, the push-button mechanism may comprise a barrel with a push-button opposite a tip end. The push-button may be connected with a depot chamber such that pressing the push-button causes the depot chamber to advance, thereby causing the depot to advance. The depot chamber may be in an incrementally slide-able relationship to the barrel, such that the depot chamber may be allowed to advance in a predetermined increment. In an example, the payload drive system may include a rotary-knock mechanism. In an aspect, the rotary-knock mechanism may include forward and rearward outer cylinders. A cam-movement converting mechanism may be stored in the forward cylinder to convert rotary movement of the rearward cylinder into an axial advancing movement. By rotating the rearward cylinder, the depot may be advanced forward to extend beyond the forward cylinder, thereby allowing a user to selectively advance and use the depot.

In some implementations, the system may include tactile and/or auditorial feedback of a payload excretion from either the supply conduit or depot conduit. In an example, the payload excretion may be the depot and/or the lift solution.

In some embodiments, the system may be used in administration of a depot into a submucosal layer of a patient's gastrointestinal tract. In some implementations, the depot may include a solid depot. In some implementations, the depot may include a liquid depot. In some embodiments, the depot may be solid and/or filamentous. In some embodiments, the depot may include one or more release mechanisms. In an example, the depot may include tunable surface erosion and/or bulk erosion properties. Examples of release mechanisms are described in Kamaly, Nazila, et al. “Degradable controlled-release polymers and polymeric nanoparticles: mechanisms of controlling drug release,” Chemical reviews 116.4 (2016): 2602-2663 herein incorporated by reference in its entirety.

In some embodiments, the depot may include a filament. In some embodiments, a filament may have a length of 10 cm, be 750 microns in diameter, and/or contain 100 mg of drug. In some embodiments, a filament may have a length of 100 cm. In some implementations, the depot may have a circular cross-section. In some implementations, the depot may have a diameter between about 0.2 to 2.0 mm.

In some embodiments, the depot may include a PLGA:PGA (Poly Lactic-co-Glycolic Acid and Poly Glycolic Acid) matrix. In some implementations, the depot may comprise a combination of PLGA and PEG. In some implementations, the depot may comprise one or more of PLA (Polylactic Acid)/PLLA/PCL (Polycaprolactone). In some embodiments the depot may be bioresorbable. In some embodiments, the depot may include a radiopaque feature configured to facilitate x-ray location.

In an aspect, the depot may be configured to be flexible/strong enough to articulate with the scope, stiff enough to be pushed by a stylet, and hard enough to not be compressed by the stylet and clog the working channel. In some implementations, the depot may be delivered at a constant delivery rate.

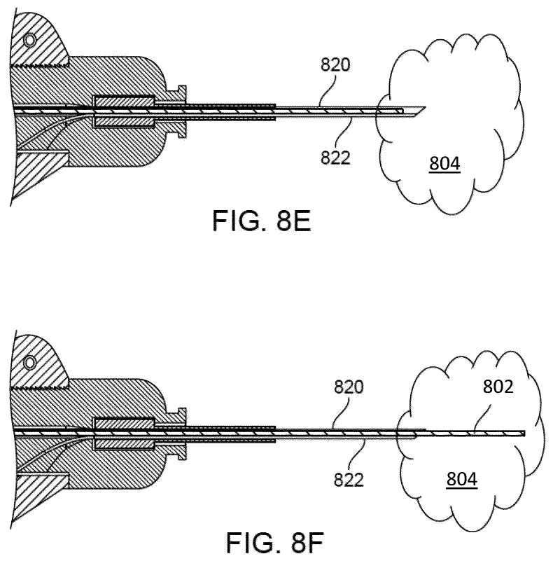

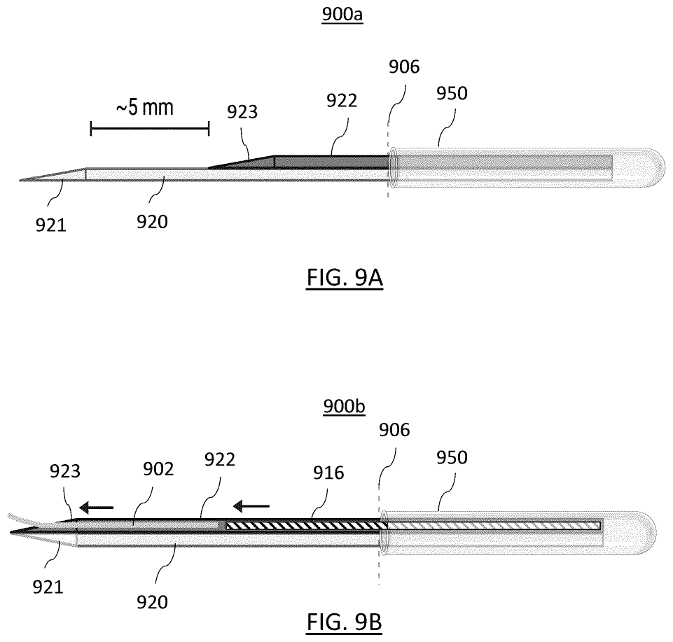

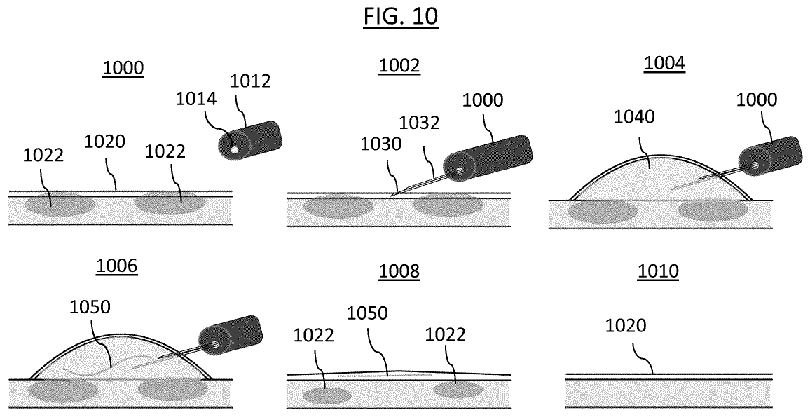

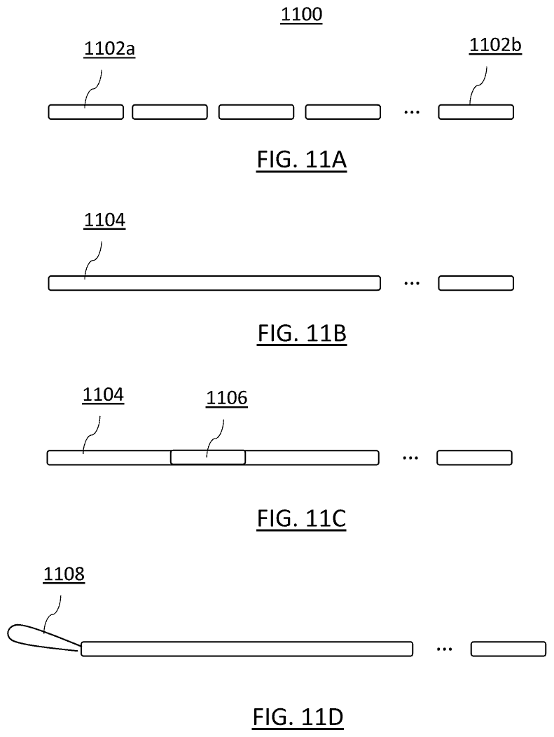

In an aspect, the depot may be configured to bend to the contours of the intestine and curl upon itself as needed within the submucosal cushion. In an example, the depot may be delivered in a series of depots 1100. In an example, each of the series of depots may be partitioned in segments 1102a-b having a length of about 1 cm (FIG. 11A). In some implementations, two or more depots of the series of depots may have differing attributes. In an example, the two or more depots of the series of depots may have differing Young's Modulus. In an example, the two or more depots of the series of depots may have differing doses of the same or different drugs. In some implementations, two or more depots of the series of depots may form a chained release of drug implant linkages. In some implementations, two or more depots of the series of depots may provide a multi-depot model or staggered filament profiles.

In an example, the depot may be delivered in a continuous filament 1104 (FIG. 11B). In an example, the continuous filament may have a length of about 8 cm but may be any other length. In an aspect, a depot partitioned in segments may have a higher Young's Modulus than a continuous filament. In an aspect, the depot may be configured to be flexible/soft enough to avoid irritation in the submucosa. In an aspect, the depot may be configured to have a Young's Modulus range of about 0.1-1 GPa. In some implementations, the continuous filament depot may have one or more segments 1106 having differing attributes (FIG. 11C). In an example, the one or more segments 1106 having differing Young's Modulus. In an example, the continuous filament depot may have certain segments having a lessor Young's Modulus such that the continuous filament depot may bend more easily without breaking. In an example, the one or more segments 1106 may have differing doses of the same or different drugs.

In some embodiments, the depot may have a payload size and/or density vary based on the desired efficiency of the delivery. In some embodiments, by administering drugs at or near a symptomatic site, a drug's bioavailability may be increased. In some embodiments, drugs may be restricted to relevant tissue. In some embodiments, absorption of the blood into the bloodstream (resulting in dilution) may be minimized. In an aspect, a dose of the drug may be reduced thereby reducing side effects. In an aspect, drug administrations may be reformulated to work with the delivery described herein including oral pills.

In some embodiments, a slow-release medication system may be inserted at the disease site. In some embodiments, a drug may start in the tissue and work “uphill” to reach the bloodstream, as opposed to the other way around. In some embodiments, this may allow patients to manage stubborn symptoms without a high dose regimen of systemic medication.

In some embodiments, the payload capacity may be larger compared to off-the-shelf endoscopic ultrasound and/or biopsy needles.

In some implementations, the depot may include one or more extra-luminal pull-tabs or retrieval loops 1108 configured to facilitate retrieval of the depot/filament (FIG. 11D). In some embodiments, a pull-tab may include a small loop that extends out of the pocket. In some embodiments, the retrieval loops may be applied using an embedding system. In an aspect, the depot may be delivered into the submucosal cushion or weal with the pull-tab extruding from the injection site.

In an aspect, the depot may be a bioresorbable formulation. In some implementations, the depot may include small molecule therapeutics, drugs, or medications. In some embodiments, the therapeutics may include anti-inflammatoires for CD, analgesics for post-operative pain relief, and/or radiotherapeutics for brachytherapy. In some embodiments, drugs may include corticosteroids. In some embodiments, corticosteroids may include budesonide and/or dexamethasone. In some implementations, the depot may be formed using protocols employed within a respective field of application. In an example, the depot may be formed protocols employed within gastroenterology or other relevant fields. In an example, a depot formulation may start with budesonide stabilized in a buffer solution (component A)+PLGA dissolved in an organic solvent (component B). In an aspect, one or more solvents may be used to facilitate formation of the PLGA depot to gradually release active drugs. In an aspect, the solvent may be selected based on having been implemented in similar applications across various indications. In an example, N-methyl-2-pyrrolidone (NMP), a leuprolide acetate for injectable suspension, (Eligard®), may be safely incorporated into an injectable formulation in small, controlled amounts. Upon injection, NMP may facilitate formation of a biocompatible PLGA depot, gradually releasing the active drug. Using controlled dosages and its rapid clearance ensures safe administration within regulatory guidelines. In an example, ethanol (DepoCyt) may be used. In an example, no solvent may be used. In some implementations, the depot may be formed using a liquid-to-gel formulation. An example of a liquid-to-gel formulation includes OncoGel™ which uses a thermosensitive PLGA-PEG-PLGA triblock copolymer that gels upon exposure to body heat to release paclitaxel. In some implementations, the depot may be formed using drug crystals which may achieve longer desired length of parenteral delivery (6+ months).

In some implementations, a depot may be formed using 25% PLGA matrix (50:50 lactide:glycolide, acid terminated), 5% PEG (plasticizer for increased flexibility and lower extrusion temp), 70% budesonide (payload API) resulting in 25 mm long filaments, 750 μm diameter, extruded at ˜100° C., weigh about 25 mg or about 17.5 mg of drug, designed to last about 6 weeks in the body. In an aspect, the depot may be configured to be bioerodible. In some implementations, a depot may be 50-70% drug by weight. In some implementations, a depot may be a homogenous mixture of drug and polymer, uniformly degrading from all sides. In some implementations, a depot may have one or more coatings or a central lumen to degrade non-uniformly.

In some implementations, the filament depots may be produced using hot melt extrusion and configured for insertion through the working channel of the endoscope. In some implementations, drug, polymer, and any additives (e.g. PEG plasticizer) may be ground and mixed together using a cryogenic mill. A machine like a Haake MiniCTW twin screw extruder may melt and extrude a polymer matrix into the filament through a circular die. The depot may include a polymer matrix, may be tuned for an indication degradation property, drug compatibility, shelf stability and sterilization, and/or mechanical properties. In an example, the indication degradation property may be specific to the submucosa environment and the targeted PKPD profile. In an example, the indication degradation property may include an elution rate limited by porosity of depot surface. In an example, the indication degradation property may include an elution rate limited by a bond strength or degradation rate of a parent matrix material or polymer.

In an example, a filament may be stiff and hard enough to not compress, bunch, and obstruct the needle channel but soft and flexible enough to curl into the fluidized submucosal cushion or weal. In an example, a filament may have a Young's Modulus of about 500 MPa. In an aspect, extra stiff filaments, if long enough, may run the risk of tunneling into the muscularis. Tunneling into the muscularis may cause an infection.

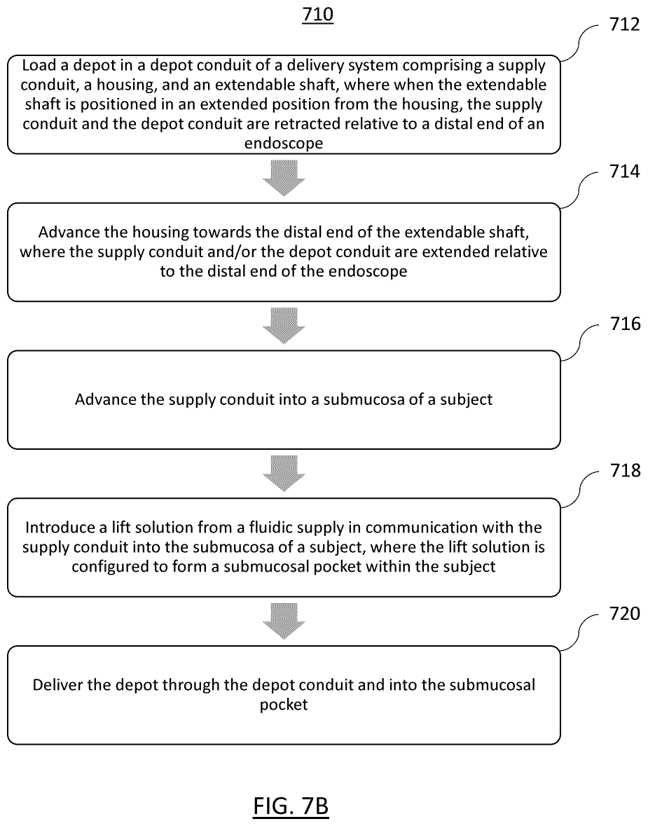

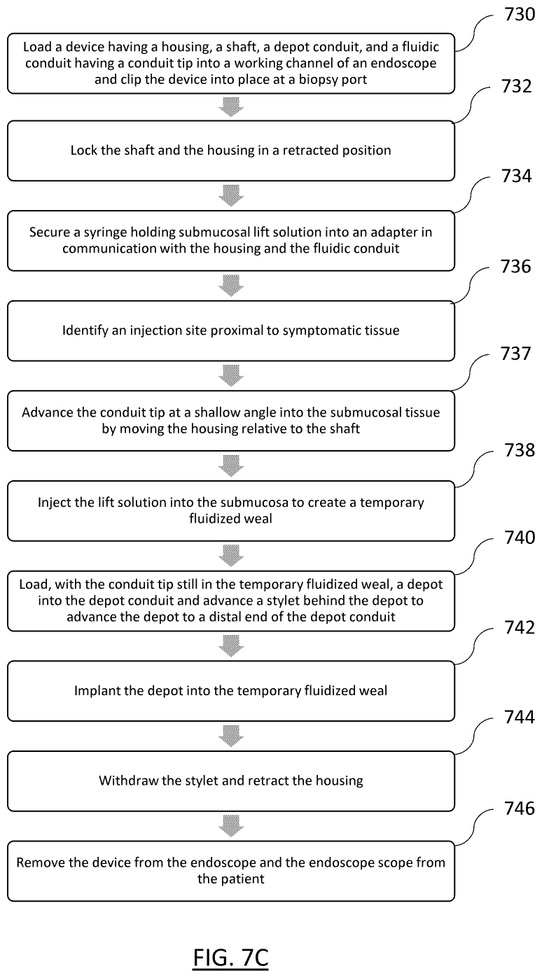

Turning to FIGS. 9A-9B, in some implementations, a delivery system 900a-b may include a supply conduit 920 and a depot conduit 922 configured to protrude from a sheath 950 extending to a distal end 906 of an endoscope. In an aspect, the supply conduit may have a conduit tip 921 and the depot conduit may have a conduit tip 923. In some implementations, the delivery system 900b may include a stylet 916 configured to advance a depot or a filament 902 through the depot conduit 922. In some implementations, the delivery system 900b may include a staggered tip design which may limit over insertion of the conduit tip 921, 923 into the weal and reduce a risk of penetrating too deep. In some implementations, a shorter filament may be used to reduce a risk of penetrating too deep. In an aspect, a moderately soft/pliable filament may be used to avoid irritation and tunneling into the intestinal tissue. In some implementations, the supply conduit and the depot conduit may be configured to protrude simultaneously from the sheath (FIG. 9B). In some implementations, the supply conduit and the depot conduit may be configured to protrude from the sheath independently from each other. In some implementations, the conduit tip may be a shape memory tip including a shape memory material. In an example, the shape memory tip may be configured for forming a curved extension from the working channel. In an aspect, the curved extension may lower an insertion angle into the submucosa and assist in encouraging longer depots or filaments to curl into a coil as the depot or filament is released. In some embodiments, the at least one of the supply conduit and the depot conduit may include a conduit tip which may be adapted to allow for easier insertion. In an example, the conduit tip may be shaped to facilitate insertion into the submucosa of the subject. In an example, the conduit tip may be flexible. In an example, the conduit tip may be curved. In some implementations, the conduit tip may include a convergent conduit tip for consolidation of payload insertion into a single perforation site.

In some implementations, the delivery system may be configured for operation with an endoscopic ultrasound scope for real-time implant guidance and/or visualization. In some implementations, the delivery system may include an ultrasound enhancing material or texturing configured to be more visible on ultrasound. In an example, the ultrasound enhancing material may include a metal or a coating at the distal end of a conduit tip or a needle. In an example, the ultrasound enhancing material may be a coating to make the conduit more hyperechoic. In an example, the endoscopic ultrasound scope may include an elevator at the distal end of the working channel. In an aspect, the elevator may allow for fine manipulation of the conduit tip and cause it to bend. In an aspect, the conduit tip is configured to be flexible and withstand bending stress applied by the elevator.

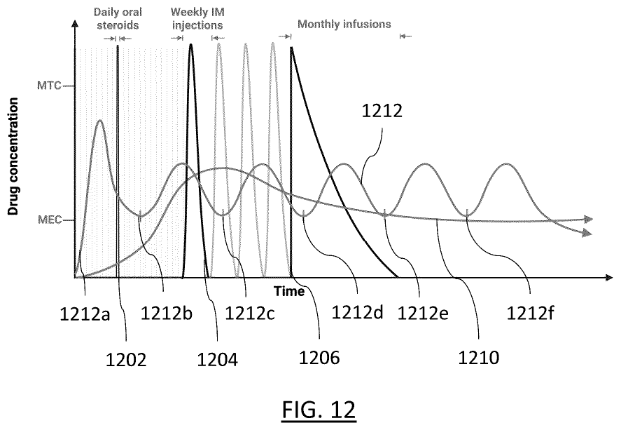

Turning to FIG. 12, a drug release cadence from the delivery system is compared relative to traditional therapeutic windows according to some implementations. Traditional therapeutic windows may be limited in their cadence within the minimum effective concentration (MEC) and the maximum therapeutic concentration or minimum toxic concentration (MTC). For example, oral steroids may have a daily cadence 1202, IM injections may have a weekly cadence 1204, and infusions may have a monthly cadence 1206. In some implementations, the delivery system may deliver a depot providing a programmed depot release cadence 1210, 1212. In an example, a programmed depot release cadence 1212a-f may have more than one crest in delivered drug concentration. In an example, the programmed depot release cadence 1212a-f may include a 6-depot chain. In an aspect, a first depot 1212a may be programmed as a larger induction dose and the depots 1212b-f may correspond with a maintenance dose of the drug. In an aspect, the depot 1212a-f may include a same drug or different drugs or different doses of the same drug. In an aspect, having a short-term depot paired with a long-term depot may enable the second depot for a larger lifespan.

Having a programmed depot release cadence may be more effective and more convenient than traditional therapeutic windows. In some implementations, the drug release cadence may be configured to release a drug on an extended time such as multiple months to a year. In some embodiments, a depot may have a payload size vary based on a controlled release timing. In some implementations, a first depot and a second depot may be deposited. In an example, the first depot and the second depot may have a different controlled release timing 1210, 1212. In some embodiments, a smaller dose may be delivered over a longer time. For example, in some embodiments, one depot may last up to 12 months. In some embodiments, sustained administration may be administered in a three month to twelve month period. In some embodiments, sustained administration may be administered in a six month to twelve month period. In an example, the depot may have a degradation time greater than three months. In an example, the depot may have a degradation time three months to three years. In some embodiments, one depot may last until the patient's next routine exam. In some embodiments, by embedding one or more drugs in a bioresorbable polymer matrix, the release rate of the one or more drugs may be controlled and/or prolonged. In some embodiments, the system may be configured for sustained and/or localized administration of the one or more drugs.

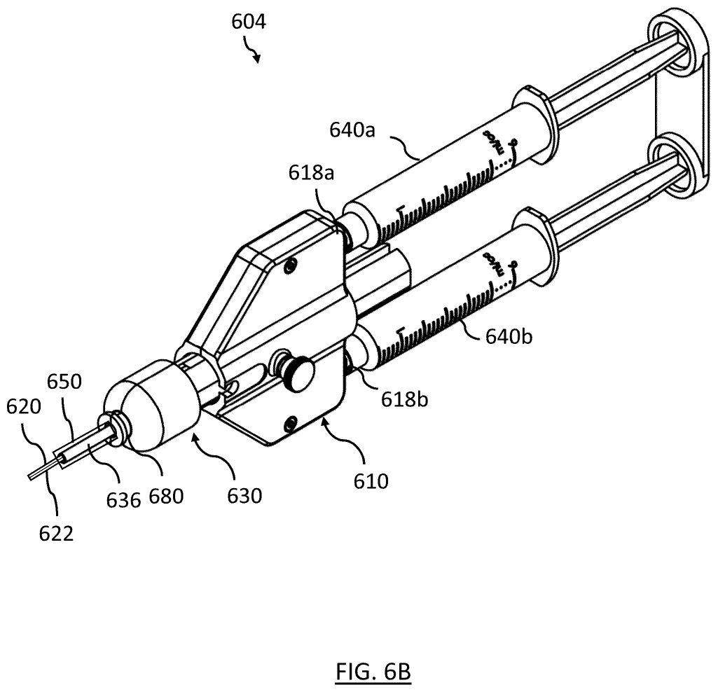

In some implementations, the delivery system may be configured to deliver a solid depot or a liquid depot. In an example, the delivery system may include a fluidic supply and a solid depot. In some implementations, the delivery system may include a delivery system including a first fluidic supply and a second fluidic supply. Turning to FIGS. 6A-6C, a delivery system 600 is shown including an endoscope 602 and a delivery system 604 configured to deliver a depot 660a-b in a submucosa layer of an intestine or gastrointestinal tract according to some implementations. In an aspect, the delivery system 604 is configured to secure at least two fluidic supplies according to some implementations. In some implementations, the delivery system 600 may be configured for depositing a fluidic depot through an endoscope in a submucosa layer of an intestine. In some embodiments, the delivery system 600 may allow for delivering and forming a conformal drug depot. In some embodiments, the delivery system 600 may allow for delivering a depot configured to cure and/or polymerize in-situ once both fluids are combined in a submucosal pocket.

In some implementations, a delivery system may include a housing 610 and an extension 630 configured to at least partially collapse within the housing, a first supply conduit 620 configured to extend through the housing and the extension, a second supply conduit 622 configured to extend through the housing and the extension. In some implementations, the first supply conduit 620 and the second supply conduit 622 may each deliver a portion of a depot.

In some implementations, the housing may be configured to secure to a first fluidic supply 640a and a second fluidic supply 640b. In an aspect, the first fluidic supply 640a may be in communication with the first supply conduit 620 configured to extend through the housing and the extension 630. In some implementations, the delivery system or housing may include a first fluidic connector 618a configured to secure to the first fluidic supply 640a and the first supply conduit 620. In some implementations, the delivery system or housing may include a second fluidic connector 618b configured to secure to the second fluidic supply 640b and the second supply conduit 622. In an aspect, the second fluidic supply 640b may be in communication with the second supply conduit 622 configured to extend through the housing and the extension 630. In some implementations, the second fluidic supply may be configured to hold at least one of a drug and an additive. In an example, the additive may modify an attribute of the submucosal pocket. In an example, the additive may include a florescent agent or tissue stain configured to make the intraintestinal or submucosal pocket visible. In an example, the additive may be configured to slow down degradation of PLGA. In an example, the additive may be a slightly acidic lift solution relative to its surroundings or PLGA. In some implementations, the pH of the lift solution may be manipulated to accelerate or decelerate the degradation or erosion of the depot's matrix material. In some implementations, the lift solution may slow down infiltration of bodily fluids into the depot or erodible matrix to slow down the degradation.

In some implementations, the lift solution may include a therapeutic function. In an example, the lift solution may include a starch or polysaccharide configured to delay absorption of the drug within the body. In some implementations, the lift solution may increase the working time for the submucosal pocket. In an example, the working time may be expanded from about 20 minutes to about an hour. In some implementations, the depot may start as a liquid and subsequently gel or solidify to conform to the unique anatomical contours of the tissue. In some implementations, the depot may require a smaller payload volume. In an example, a 50 mg filament depot may be replaced with a 5 g gel depot.

In some implementations, the first fluidic supply may comprise a first agent 660a and the second fluidic supply may comprise a second agent 660b. In some implementations, the first agent and the second agent may be configured for in-situ curing of a submucosal drug depot. In some implementations, the first agent and the second agent may be configured to interact within the intraintestinal or submucosal pocket and change a property of at least one of the first agent and the second agent. In some implementations, the first agent and the second agent may be configured to utilize polymer precipitation. In an example, a chemical embodiment includes PLGA+N-methyl-2-pyrrolidone (NMP). In an aspect, the PLGA is configured to dissolve in NMP, and upon injection, water exchange causes phase separation, forming a depot. In an example, injectable depots may include those produced by Eligard and Atridox. In some implementations, the first agent and the second agent may be configured to use or form hydrogels. In an example, the first agent and the second agent may include Alginate and Calcium Chloride (CaCl₂). In an example, ionic crosslinking of alginate with divalent calcium ions may be used to form a stable hydrogel.

In some implementations, a multi-fluidic supply delivery system may include independently triggered/operable payload conduit or channels. In some implementations, a multi-fluidic supply delivery system may include a brace 690 forming tethered triggered/operable payload conduits or channels. In some implementations, a multi-fluidic supply delivery system may be configured for injecting both solid and liquid payloads into submucosal tissue. In an example, the depot conduit may be configured to deliver a solid payload and the first and/or the second supply conduits may be configured to deliver a liquid payload. In some implementations, the system may include a sheath 650 configured to enclose at least one of the supply conduit 620 or the depot conduit 622, or both. In an example, the sheath 650 may connect at a distal end of the extension 630 and extend to a distal end 606 of the endoscope. In some embodiments, the system 604 may include a releasable endoscope connector 680 configured to mate to standard endoscope and/or colonoscope instrument channel ports.

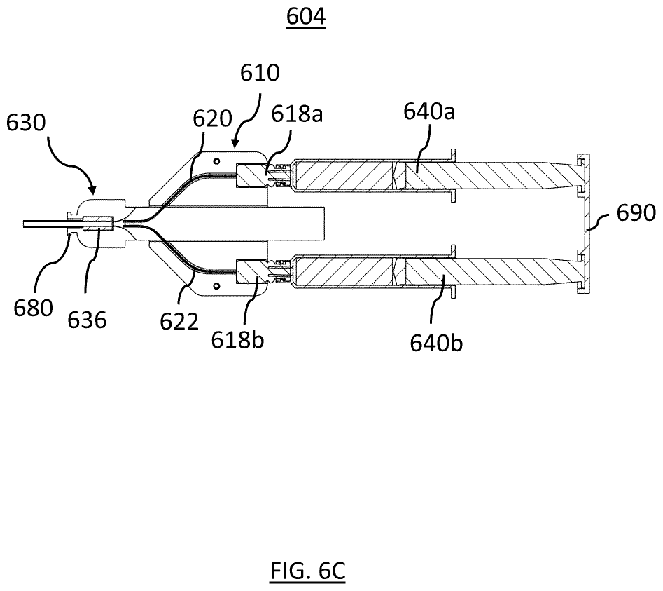

In some implementations, a method for delivering a depot into a submucosa of a subject may include advancing a portion of a delivery system through a working channel of an endoscope and securing the delivery system to the endoscope. Turning to FIG. 7A, disclosed herein is a method 700 for delivering a depot into a submucosa of a subject, comprising a step 702 of loading a depot in a depot conduit of a delivery system comprising a supply conduit, a housing, and an extension, advancing the depot conduit and the supply conduit approximate an insertion site at submucosa of the subject, a step 704 of advancing a distal end of the supply conduit into a portion of submucosa of the subject, a step 706 of forming an intraintestinal or submucosal pocket within the subject, and a step 708 of delivering a depot through the depot conduit and into the submucosal pocket. In some implementations, the method may include identifying an implant site. In an example, identifying an implant site may include one or more imaging modalities. In an example, a camera may be used to identify the implant site and/or the distal end of the supply conduit. In an example, ultrasound guidance may be used to identify the implant site and/or the distal end of the supply conduit. In an example, advancing the depot conduit and the supply conduit approximate an insertion site at submucosa of the subject may include advancing to an extension working distance of the supply conduit and depot conduit. In an example, the extension working distance of the supply conduit and depot conduit may be about 1-5 cm beyond the end of the endoscope, working channel, and/or the sheath.

In some implementations, advancing the distal end of the supply conduit into the portion of submucosa of the subject includes moving the housing relative to the extension. In some implementations, the method may include delivering the depot without removing the distal end of the supply conduit from the submucosa of the subject. In some implementations, forming the intraintestinal or submucosal pocket within the subject includes delivering a lift solution through the supply conduit into the submucosa of the subject. In some implementations, the supply conduit may be configured to deliver the lift solution within a fluidic supply secured to the housing. In some implementations, the depot may be delivered using a stylet. In some implementations, the depot may be delivered using a second fluidic supply secured to the housing. In some implementations, the fluid cushion may be configured to be retained for about 10 minutes. In some implementations, the fluid cushion may be configured to be retained for about one (1) hour. In some implementations, the method may include releasing a trigger configured to resist movement between the extension and the housing.

Turning to FIG. 7B, disclosed herein is a method 710 for delivering a depot into a submucosa of a subject, comprising a step 712 of loading a depot in a depot conduit of a delivery system comprising a supply conduit, a housing, and an extension, where when the extension is positioned in an extended position from the housing, the supply conduit and the depot conduit are retracted relative to a distal end of an endoscope, a step 714 of advancing the housing towards the distal end of the extension, where the supply conduit and/or the depot conduit are extended relative to the distal end of the endoscope, a step 716 of advancing the supply conduit into the submucosa of a subject, a step 718 of introducing a lift solution from a fluidic supply in communication with the supply conduit into the submucosa of a subject, where the lift solution is configured to form an intraintestinal or submucosal pocket within the subject, and a step 720 of delivering the depot through the depot conduit and into the submucosal pocket.

Turning to FIG. 7C, disclosed herein is a method for delivering a depot into a submucosa of a subject, comprising a step 730 of loading a delivery system having a housing, an extension, a depot conduit, and a fluidic conduit having a conduit tip into a working channel of an endoscope and clip the delivery system into place at a biopsy port, a step 732 of locking the extension and the housing in a retracted position, a step 734 of securing a fluidic supply holding submucosal lift solution into an adapter in communication with the housing and the fluidic conduit, a step 736 of identifying an injection site proximal to symptomatic tissue, a step 737 of advancing the conduit tip at a shallow angle into the submucosal tissue by moving the housing relative to the extension, a step 738 of injecting the lift solution into the submucosa to create a temporary fluidized weal, a step 740 of loading with the conduit tip still in the temporary fluidized weal, a depot into the depot conduit by advancing a stylet behind the depot to advance the depot to a distal end of the depot conduit, a step 742 of implanting the depot into the temporary fluidized weal, a step 744 of withdrawing the stylet and retract the housing, and a step 746 of removing the delivery system from the endoscope and the endoscope scope from the subject.

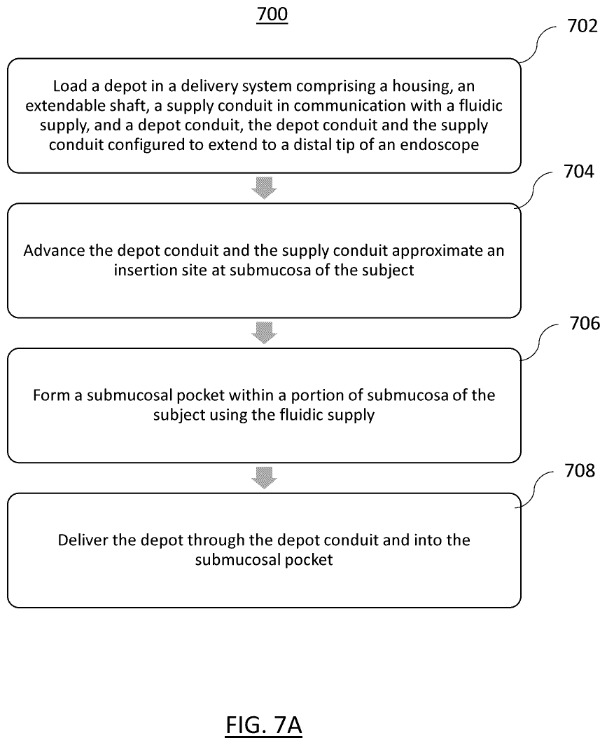

FIGS. 8A-8F illustrate a series of stages in a method of delivering a depot according to some implementations. In some implementations, the method includes advancing a portion of a delivery system through a working channel of an endoscope and securing the delivery system to the endoscope. In an example, the delivery system may comprise a supply conduit 820, a housing 810, and an extension 830, wherein the extension is positioned in an extended position from the housing, wherein the supply conduit and the depot conduit are retracted relative to the distal end of an endoscope. In some implementations, the method includes a step 800a of loading a depot in a depot conduit 822 of the delivery system. In some implementations, the method includes a step 800b of disengaging a trigger configured to prevent movement of at least one of the housing and the extension. In some implementations, the method includes a step 800c of advancing the housing towards the distal end of the extension, where the supply conduit and/or the depot conduit are extended relative to a distal end of an endoscope. In some implementations, the method includes a step of advancing a distal end of the supply conduit into a submucosa of a subject. In some implementations, the method includes a step of advancing a distal end of the first and second supply conduits into a submucosa of a subject. In some implementations, the method includes a step 800d of introducing a lift solution from a fluidic supply into a submucosa of a subject, where the lift solution may be configured to form an intraintestinal or submucosal pocket 804 within the subject. In some implementations, the method includes a step 800f of delivering a depot 802 through the depot conduit and into the intraintestinal or submucosal pocket within the subject. In some implementations, the step of delivering a depot through the depot conduit and into the intraintestinal or submucosal pocket within the subject is done without removing the distal end of the supply conduit from the submucosa of the subject.

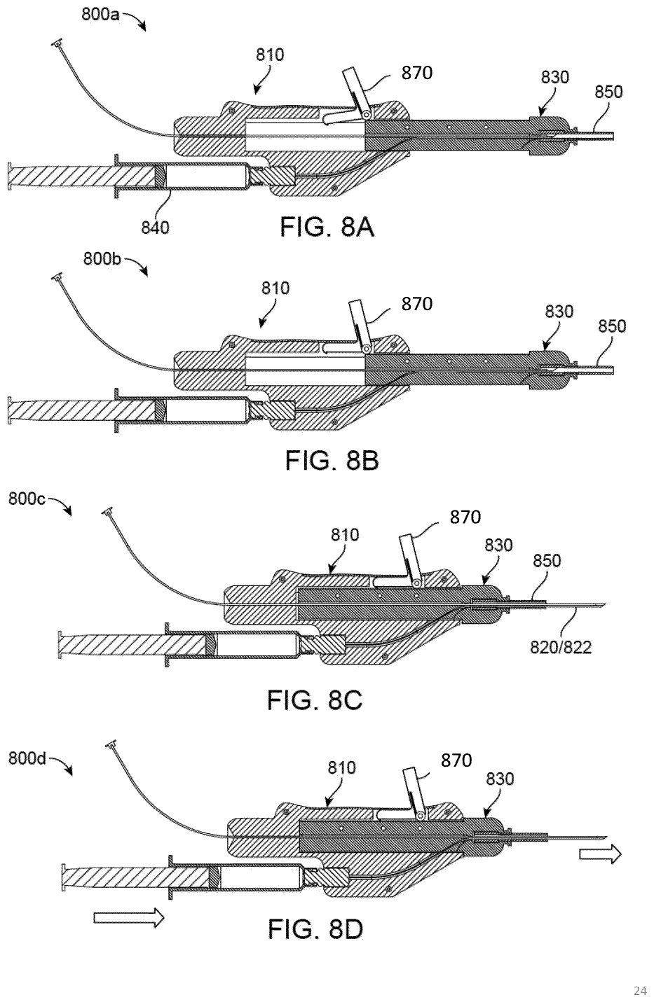

FIG. 10 illustrates a series of stages in a method of delivering a depot according to some implementations. In some implementations, the method includes securing a delivery system having a supply conduit and a depot conduit within an endoscope 1012 having a working channel 1014. In some implementations, the method includes a step 1000 of advancing a distal end of the endoscope 1012 approximate a submucosa 1020 of a subject approximate a tissue of interest 1022. In some implementations, the method includes a step 1002 of advancing a distal end of the supply conduit into a submucosa of a subject. In some implementations, the method includes a step of advancing a distal end of the first and second supply conduits into a submucosa of a subject. In some implementations, the method includes a step 1004 of introducing a lift solution from a fluidic supply into a submucosa of a subject, where the lift solution may be configured to form an intraintestinal or submucosal pocket 1040 within the subject. In some implementations, the method includes a step 1006 of delivering a depot 1050 through the depot conduit and into the intraintestinal or submucosal pocket within the subject. In some implementations, the step of delivering the depot through the depot conduit and into the intraintestinal or submucosal pocket within the subject is done without removing the distal end of the supply conduit from the submucosa of the subject. In some implementations, the method includes a step 1008 of removing the delivery system and leaving behind the depot 1050 approximate the tissue of interest 1022. In some implementations, the method includes a step 1010 of treating an underlying condition approximate the submucosa 1020.

### Experimental Results

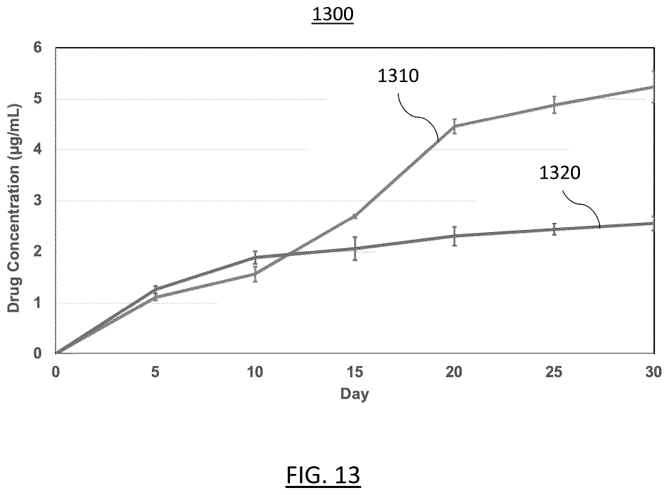

A benchtop degradation study of two depot types was conducted to determine release kinetics of small molecule medication embedded in a depot. Samples included a first depot type and a second depot type having filaments approximately 2 cm long and approximately 20% budesonide (by wt.). Both depots were formulated with an acid-terminated PLGA blend (50:50 lactide:glycolide). Samples were submersed in a PBS solution, heated to 37° C., and agitated. Drug concentrations were collected from the samples in 5-day intervals and tested via High-performance liquid chromatography (HPLC). FIG. 13 shows a graph 1300 of average drug concentration (ug/mL) with standard deviation (bars). Concentration curve 1320 correlates to a batch of the first depot type having ˜650 μm-thick filaments (˜15 mg each) and concentration curve 1310 correlates to a batch of the second depot type having ˜900 μm-thick filaments (˜20 mg each). Notably, the thicker filament or second depot type eluted a larger amount of drug in the bulk degradation phase and indicates the drug eluding will last longer. In both cases, sustained release after 20+ days was rising and the concentration rate was relatively consistent.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

What is claimed is:

1. A system for delivering a depot within a submucosal pocket, the system comprising:
a housing including a fluidic connector configured to secure to a fluidic supply;
an extension configured to at least partially collapse within the housing;
a supply conduit configured to extend through the housing and the extension, the supply conduit configured to deliver a lift solution configured to form a submucosal pocket within a subject; and
a depot conduit configured to extend through the housing and the extension and to deposit a depot within the submucosal pocket;
wherein when the extension is configured to at least partially collapse within the housing, whereby at least one of the supply conduit and the depot conduit advance distally past a distal end of an endoscope.

2. The system of claim 1, further comprising a sheath connected to a distal end of the extension, wherein, when the extension at least partially collapses within the housing, the at least one of the supply conduit and the depot conduit advance past a distal end of the sheath.

3. The system of claim 1, further comprising a trigger configured to secure the extension relative to the housing.

4. The system of claim 1, wherein the housing includes at least one of a supply conduit recess and a depot conduit recess.

5. The system of claim 4, wherein the supply conduit recess is configured to at least partially direct the supply conduit towards an extension lumen of the extension and/or a distal connector.

6. The system of claim 5, wherein the depot conduit recess is configured to at least partially direct the depot conduit towards an extension lumen of the extension and/or a distal connector.

7. The system of claim 1, further comprising an endoscope connector configured to secure the delivery system to the endoscope.

8. The system of claim 1, wherein the supply conduit is concentric with the depot conduit.

9. The system of claim 1, wherein a distal tip of the supply conduit is staggered from the distal tip of the depot conduit.

10. The system of claim 1, wherein the depot is a series of depots configured to advance inline.

11. The system of claim 1, wherein the depot is a continuous filament configured to curl within the submucosal pocket.

12. The system of claim 1, wherein the depot includes one or more extra-luminal pull-tabs.

13. The system of claim 1, wherein the depot includes two or more depots having differing Young's Modulus.

14. The system of claim 1, wherein the depot includes two or more depots having differing doses of a same drug or a different drug.

15. The system of claim 1, wherein the depot includes a programmed degradation window of greater than 3 months.

16. The system of claim 1, wherein the depot includes a programmed depot release cadence having more than one crest in delivered drug concentration.

17. The system of claim 1, wherein the housing includes a second fluidic supply configured to hold at least one of a drug and an activation agent.

18. The system of claim 1, further comprising a loading chamber configured to receive the depot or a depot cartridge.

19. The system of claim 1, wherein the fluidic supply is a syringe.

20. A method for delivering a depot into a portion of submucosa of a subject, the method comprising:
loading a depot in a delivery system comprising a supply conduit, a depot conduit, a housing, and an extension, the depot conduit and the supply conduit being configured to extend to a distal tip of an endoscope;
advancing the depot conduit and the supply conduit approximate an insertion site at the portion of submucosa of the subject;
advancing a distal end of the supply conduit into a portion of submucosa of the subject;
forming a submucosal pocket within the subject; and
delivering the depot through the depot conduit and into the submucosal pocket.

21. The method of claim 20, wherein the depot conduit and the supply conduit are advanced through a sheath connected to a distal end of the extension.

22. The method of claim 20, wherein delivering the depot is done without removing the distal end of the supply conduit from the submucosa of the subject.

23. The method of claim 20, wherein forming the submucosal pocket within the subject includes delivering a lift solution through the supply conduit into the submucosa of the subject.

24. The method of claim 20, wherein the supply conduit is configured to deliver the lift solution within a fluidic supply secured to the housing.

25. The method of claim 20, wherein the lift solution is configured to be retained for about 1 hour.

26. The method of claim 20, further comprising releasing a trigger configured to releasably secure the extension to the housing.

27. A method for delivering a depot into a submucosa of a subject, the method comprising:
loading a depot in a delivery system comprising a housing, an extension, a supply conduit in communication with a fluidic supply, and a depot conduit, the depot conduit and the supply conduit configured to extend to a distal tip of an endoscope;
advancing the depot conduit and the supply conduit approximate an insertion site at submucosa of the subject by moving the housing relative to the extension;
forming a submucosal pocket within a portion of submucosa of the subject using the fluidic supply; and
delivering the depot through the depot conduit and into the submucosal pocket.

28. A system for delivering a subcutaneous depot, the system comprising:
an endoscope comprising a working channel;
a fluidic supply; and
a delivery system for delivering a depot within a submucosal pocket, the delivery system comprising:
a housing including a fluidic connector configured to secure to the fluidic supply,
an extension configured to at least partially collapse within the housing,
a supply conduit configured to extend through the housing and the extension, the supply conduit configured to deliver a lift solution configured to form a submucosal pocket within a subject, and
a depot conduit configured to extend through the housing and the extension and to deposit a depot within the submucosal pocket,
wherein, when the extension at least partially collapses within the housing, at least one of: the supply conduit, the depot conduit, or both the supply conduit and the depot conduit advance distally past a distal end of the endoscope.

29. The method of claim 27, wherein delivering the depot through the depot conduit and into the submucosal pocket within the subject is done without removing the distal end of the supply conduit from the submucosa of the subject.
